SEQUENCE LISTING (1 sequence)

SEQ ID NO: 1 - DNA, 1382 bp, Bifidobacterium animalis
  source 1..1382: mol_type = genomic DNA; sub_species = lactis
cgaaacatccctaggacagcttgctgtcggggtgagagtggcgaacgggtgagtaatgcgtgaccaacctgccctgtgcaccggaatagctcctggaaacgggtggtaataccggatgctccgctccatcgcatggtggggtgggaaatgcttttgcggcatgggatggggtcgcgtcctatcagcttgttggcggggtgatggcccaccaaggcgttgacgggtagccggcctgagagggtgaccggccacattgggactgagatacggcccagactcctacgggaggcagcagtggggaatattgcacaatgggcgcaagcctgatgcagcgacgccgcgtgcgggatggaggccttcgggttgtaaaccgcttttgttcaagggcaaggcacggtttcggccgtgttgagtggattgttcgaataagcaccggctaactacgtgccagcagccgcggtaatacgtagggtgcgagcgttatccggatttattgggcgtaaagggctcgtaggcggttcgtcgcgtccggtgtgaaagtccatcgcctaacggtggatctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggccctttccacgggtcccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgtgccggatcgccgtggagacacggtttcccttcggggccggttcacaggtggtgcatggtcgtcgtcagctcgtgtcgtgagatgttgggttaagtcccgcaacgagcgcaaccctcgccgcatgttgccagcgggtgatgccgggaactcatgtgggaccgccggggtcaactcggaggaaggtggggatgacgtcagatcatcatgccccttacgtccagggcttcacgcatgctacaatggccggtacaacgcggtgcgacacggtgacgtggggcggatcgctgaaaaccggtctcagttcggatcgcagtctgcaactcgactgcgtgaaggcggagtcgctagtaatcgcggatcagcaacgccgcggtgaatgcgttcccgggccttgtacacaccgcccgtcaagtcatgaaagtgggtagcacccgaagccggtggccgcgac

(19) United States
(12) Patent Application Publication
(10) Pub. No.: US 2025/0332203 A1
(43) Pub. Date: Oct. 30, 2025

(54) BIFIDOBACTERIUM ANIMALIS SUBSP. LACTIS GOLDGUT-BB21 AND APPLICATION THEREOF

(71) Applicant: SHENZHEN PORSHEALTH BIOENGINEERING CO., LTD., SHENZHEN (CN)

(72) Inventors: SILU ZHANG, SHENZHEN (CN); YANYI ZHENG, SHENZHEN (CN); GUOXUN XIAO, SHENZHEN (CN); ZHIZHU ZHANG, SHENZHEN (CN); XIAOLI ZHOU, SHENZHEN (CN)

(21) Appl. No.: 19/008,906

(22) Filed: Jan. 3, 2025

(30) Foreign Application Priority Data
Jan. 5, 2024 (CN) 202410024101.6

(51) Int. Cl. A61K 35/745; A61P 1/14; C12N 1/20; C12R 1/01

(52) U.S. Cl. CPC A61K 35/745; A61P 1/14; C12N 1/205; C12R 2001/01

(57) ABSTRACT

The present invention belongs to the technical field of biomedicine. The present invention provides a Bifidobacterium animalis subsp. lactis GOLDGUT-BB21, which has been preserved at China General Microbiological Culture Collection Center, abbreviated as CGMCC, on Dec. 18, 2023. The preservation number is: CGMCC No.29347, and the preservation address is: Building 3, Courtyard 1, Beichen West Road, Chaoyang District, Beijing. The strain of the present invention has the functions of treating chronic fatigue syndrome and irritable bowel syndrome.

### SEQUENCE LISTING

The sequence listing was submitted as a XML file filed via EFS-Web, with a file name of “Sequence-Listing.XML”, a creation date of Jan. 2, 2025, and a size of 3,544 bytes. A replacement of the sequence listing is submitted as a XML file filed via EFS-Web, with a file name of “Sequence-Listing-v2. XML”, a creation date of Feb. 10, 2025, and a size of 3,521 bytes. The replacement of the sequence Listing filed via EFS-Web is a part of the specification and is incorporated in its entirety by reference herein.

### TECHNICAL FIELD

The present invention belongs to the technical field of biomedicine.

### BACKGROUND

Chronic fatigue syndrome (CFS) is a complex multisystem debilitating disease which is easy to be ignored, and is mainly manifested by severe fatigue, cognitive impairment and gastrointestinal disorders. The diagnostic criteria and the pathogenic mechanism of CFS are still unclear so far, and there is no targeted specific drug [1-2].

Patients with CFS are often accompanied by irritable bowel syndrome (IBS). IBS is a common chronic intestinal disorder in clinical practice, and includes the main symptoms of abdominal pain, abdominal discomfort and changes in defecation function, which have a great impact on the lives of people [3-5].

The existing treatment strategies are often limited to the treatment of corresponding symptoms (anti-fatigue, analgesic and anti-anxiety drugs), and have limited efficacy and certain side effects. It is difficult to have an effective treatment plan for all patients due to the heterogeneity of patients.

### SUMMARY

In order to effectively improve or treat CFS and IBS, a first purpose of the present invention is to provide a Bifidobacterium animalis subsp. lactis GOLDGUT-BB21, which has been preserved at China General Microbiological Culture Collection Center, abbreviated as CGMCC, on Dec. 18, 2023. The preservation number is: CGMCC No.29347, and the preservation address is: Building 3, Courtyard 1, Beichen West Road, Chaoyang District, Beijing.

In specific embodiments of the present invention, the present invention further provides an application of the Bifidobacterium animalis subsp. lactis GOLDGUT-BB21 in preparation of health food or drugs for treating or improving CFS.

In specific embodiments of the present invention, further, the CFS includes: symptoms of inhibited weight gain, impaired spatial memory, and impaired motion ability.

In specific embodiments of the present invention, the present invention further provides an application of the Bifidobacterium animalis subsp. lactis GOLDGUT-BB21 in preparation of health food or drugs for treating or improving IBS.

In specific embodiments of the present invention, the present invention fourthly provides the IBS including symptoms of inhibited weight gain, visceral hypersensitivity, anxiety, and intestinal wall barrier dysfunction.

In specific embodiments of the present invention, the present invention fifthly provides an application of the Bifidobacterium animalis subsp. lactis GOLDGUT-BB21 in preparation of bacterial agents for inhibiting Escherichia coli, Staphylococcus aureus or Pseudomonas aeruginosa.

The Bifidobacterium animalis subsp. lactis GOLDGUT-BB21 in the present invention can improve the memory and cognitive functions of CFS hosts, enhance the motor coordination ability of the hosts, can also improve the visceral hypersensitivity and anxiety state of IBS hosts and can improve the intestinal barrier function of the hosts.

### DESCRIPTION OF DRAWINGS

FIG. 1 is a diagram of colony morphology of Bifidobacterium animalis subsp. lactisGOLDGUT-BB21 strain.

FIG. 2 is a sample diagram of immunohistochemical results of the colon tissue of Bifidobacterium animalis subsp. lactis GOLDGUT-BB21 strain in a treatment experiment of IBS mice.

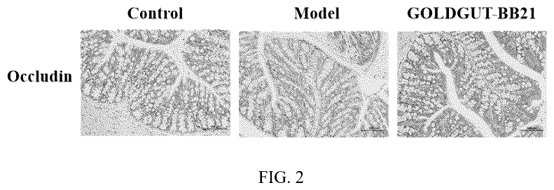

### DETAILED DESCRIPTION

### Embodiment

Modified MRS liquid medium (g/L): 52.4 g of MRS broth, 0.01 g of hemin, 0.001 g of resazurin and 10% (v/v) of clarified rumen fluid are included; distilled water is added to 1 L; pH is 7.2±0.1; and the medium is sterilized at 115° C. for 25 min, and then used after high pressure sterilization. 15 g of additional agar is added to a solid medium.

### 1. Acquisition, Isolation and Identification of Strain

The strain was obtained by anaerobic separation of fresh feces provided by a healthy fecal donor (that signs the informed consent) by the patent inventor in Qingdao, Shandong Province on Jan. 17, 2022.

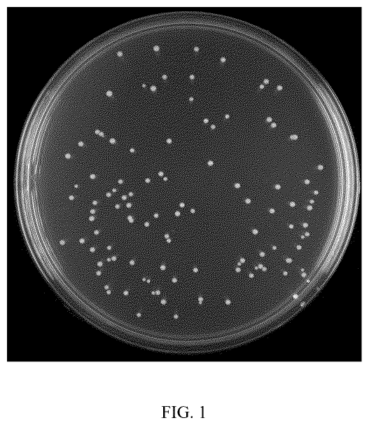

Colony morphology: The modified MRS broth medium per liter comprises: 52.4 g of MRS broth, 0.01 g of hemin, 0.001 g of resazurin and 10% (v/v) of clarified rumen fluid; distilled water is added to 1 L; pH is 7.2±0.1; and the medium is sterilized at 115° C. for 25 min, and then used after high pressure sterilization. 15 g of additional agar is added to a solid medium. After anaerobic culture at 37° C. for 2-3 days, the colony has a diameter of 2-3 mm, is white and smooth, and has no water-soluble pigment (FIG. 1).

Molecular identification method: Through the complete sequence analysis of 16S rRNA, the strain is identified and classified as Bifidobacterium animalis subsp. lactis. The strain is named GOLDGUT-BB21. The strain has been preserved at China General Microbiological Culture Collection Center (abbreviated as CGMCC) on Dec. 18, 2023. The preservation number is: CGMCC No.29347, and the preservation address is: Building 3, Courtyard 1, Beichen West Road, Chaoyang District, Beijing. That is, the preservation number of the patent strain is CGMCC No.29347. The 16S rRNA sequence homology with Bifidobacterium animalis subsp. lactis standard strain is 99%, and the sequence is shown by SEQ ID NO.1 in the sequence listing.

### 2. Culture and Preservation Methods

The Bifidobacterium animalis subsp. lactis is activated twice in the modified MRS broth medium, then inoculated into a modified MRS liquid medium with the same composition at an inoculation amount of 0.1%-10%, and cultured anaerobically at 37° C. for 2 days.

Long-term preservation method of bacteria: The bacterial solution is centrifuged at 4° C. and 10000 rpm for 2 minutes to collect the precipitated cells; the cells are washed with PBS, then suspended by 5:1 in a solution containing 15%-25% of skimmed milk powder, freeze-dried and then preserved hermetically, or frozen in liquid nitrogen in a preservation solution containing 15% of glycerin and 85% of serum and then preserved at −80° C.

### 3. Use and Effect of the Strain

3.1 Screening of Bifidobacterium animalis subsp. lactis GOLDGUT-BB21 strain and animal experiment on alleviating CFS

### 3.1.1 Materials and Methods:

1) Modified MRS Liquid Medium (g/L):

The modified MRS liquid medium comprises 52.4 g of MRS broth, 0.01 g of hemin, 0.001 g of resazurin and 10% (v/v) of clarified rumen fluid; distilled water is added to 1 L; pH is 7.2±0.1; and the medium is sterilized at 115° C. for 25 min, and then used after high pressure sterilization. 15 g of additional agar is added to a solid medium.

2) Preparation Method of Bifidobacterium animalis subsp. lactis GOLDGUT-BB21 Powder Feed

The Bifidobacterium animalis subsp. lactis is activated twice in the modified MRS broth medium, then inoculated into a modified MRS liquid medium with the same composition at an inoculation amount of 0.1%-10%, and cultured anaerobically at 37° C. for 2 days. The bacterial solution is centrifuged at 4° C. and 10000 rpm for 2 minutes to collect the precipitated cells; the cells are washed with PBS, then suspended by 5:1 in a solution containing 15%-25% of skimmed milk powder, freeze-dried, tested for viable count and mixed into the feed.

### 3) Test for Acid and Alkali Tolerance

Anaerobic PBSs (8 mM of Na₂HPO₄, 136 mM of NaCl, 2 mM of KH₂PO₄, and 2.6 mM of KCl) with pH of 3, 3.5, 4, 7, 9 and 10 are prepared, with a total of 6 gradients, wherein the PBS with pH of 7 is a control group. The seed solution of each plant is packaged into 1.5 mL EP tubes at 0.1 mL/tube, and centrifuged at 6000 rpm for 5 min to remove the supernatant; and 3 parallels are set for each plant. The bacteria are resuspended by 1 mL of PBS with different pH, and incubated at 37° C. for 4 h. The bacterial suspension is diluted gradiently with the PBS of pH of 7 to balance the pH value of the bacterial suspension. 50 μL of bacterial suspension is coated in the MRS solid medium, and cultured at 37° C. for 24-48 h. Counting is performed after a visible colony is formed, and the survival rate is calculated.

### 4) Utilization of Carbon Sources and Verification of Oxidation

Utilization of different carbon sources and the oxidation of Bifidobacterium animalis subsp. lactis GOLDGUT-BB21 are tested by AN Biolog microplate. The nutrients and biochemical reagents used by the AN microplate are shown in Table 4. The method is as follows: 1 ml of Bifidobacterium animalis subsp. lactis GOLDGUT-BB21 bacterial solution at the end of logarithmic growth is absorbed, centrifuged to remove the supernatant, washed with the seed solution for 3 times, and suspended with 1 ml of seed solution. 50 μL of bacterial solution is diluted in 10 ml of seed solution, mixed evenly, then packaged into a Biolog plate (100 μL/well) and incubated anaerobically at 37° C. for 24 h. After culture, OD₅₉₀ and OD₇₅₀ are detected by a microplate reader respectively, and the values are recorded. The utilization of carbohydrates, organic acid and other substances of Bifidobacterium animalis subsp. lactis GOLDGUT-BB21 are calculated, and a calculation method is: Y=OD₅₉₀−OD₇₅₀.

### 5) Test for Bacteriostatic Ability

The Bifidobacterium animalis subsp. lactis GOLDGUT-BB21 is inoculated into an anaerobic glass tube of the modified MRS liquid medium at 1% (v/v) and statically cultured at constant temperature of 37° C. for 12 h. Pathogenic strains such as Escherichia coli, Staphylococcus aureus and Pseudomonas aeruginosa are inoculated in the liquid LB medium respectively, and cultured overnight at 37° C. and 250 rpm in a constant-temperature shaker. Then, the bacterial suspension is prepared. The MRS solid medium is cooled to about 55° C. and mixed evenly with the pathogenic bacterial suspension in a certain proportion; the viable count of pathogens in the system is 10⁶ CFU/mL; then the suspension is quickly poured into a plate in which an Oxford cup is placed in advance; and the Oxford cup is removed after the medium is cooled and solidified. 200 μL of Bifidobacterium animalis subsp. lactis GOLDGUT-BB21 fermentation broth is injected into each well. The plate is lightly covered and then uprightly placed in a constant temperature incubator at 37° C. After culture for 24 h, the plate is observed, and the diameter of an antibacterial zone is measured with a vernier caliper.

### 6) Metabolite Detection

Firstly, ethyl acetate is added at 1:1 equal volume for extraction. Then, vortex oscillation is conducted at 15 min/time, with a standing interval of 2 min; the solution is centrifuged at 10000 rpm for 10 min. The supernatant is absorbed and filtered through an organic filtration membrane. 300-400 μL of organic phase is determined by GC/MS analysis.

### 7) Antibiotic Sensitivity

The antibiotic sensitivity of Bifidobacterium animalis subsp. lactis GOLDGUT-BB21 is determined by K-B method (Kirby-Bauer).

### 8) Design of Animal Experiment

SPF wild female C57BL/6J mice of 4-6 weeks old are adaptively fed for one week in the experiment.

Animal adaptation: Before the formal start of the experiment, the mouse models (except the control group) are put into an interference box of a sleep deprivation instrument for adaptation, for 3 consecutive days, and 3 hours a day.

Modeling and intervention: From the first week to the end of the experiment, each mouse in GOLDGUT-BB21 group is given mixed quantitative bacterial meal feed (viable count 2×10⁹ CFU/day/mouse); and the mice in the control group and the model group are given aseptic meal feed every day.

On weeks 2-10 after the start of the experiment, all the mice in the model group and the GOLDGUT-BB21 group are subjected to sleep deprivation, with sleep deprivation and light disturbance for 23 h and rest for 1 h every day.

### 9) Index Detection

Behavioral detection: elevated plus maze test; Y-maze spontaneous alternation experiment; Y-maze new arm recognition experiment; rotarod test

Elevated plus maze test: The anxiety-like behaviors of the mice are assessed. The room is quiet and dark. The mice are put into the maze from the central region and face the closed arm. The activities within 6 min are recorded. Observation indexes are recorded by an automatic camera system and a computer analysis and processing system: the number of entry in the open arm and the motion distance of the open arm are used as the assessment indexes.

Y-maze spontaneous alternation experiment: The memory and cognitive abilities of the mice are assessed. The mice are placed from the end of one arm and allowed to freely explore for 8 min. The experimenter is out of sight of the mice. Motion trajectories are tracked by the camera, and the sequence numbers of each arm in which the mice come are recorded in sequence. The total number of entry in channels, the total motion distance and the spontaneous alternation rate are calculated. Spontaneous alternation rate (%)=total number of transformations of a triple string with the sequence numbers of all three arms/possible number of transformations for entry in the maze arms (the total number of entry in the arms−2)×100%.

Y-maze new arm recognition experiment: The memory ability of the mice is assessed by using the instinct of the animal to explore new fields. The experiment is divided into two parts. That is, the first stage of the experiment is a training period, and the second stage is a detection period. A new different arm is shielded by a baffle plate in the first stage of the experiment, that is, during the training period, and is opened in the second stage, that is, during the detection period. A starting arm is an arm in which the mice enter the maze. The starting arm and other arms are open throughout the experiment, and are used for the animals to enter and exit freely. The camera is placed at 1.5 m above the maze and used for shooting and recording the whole process. During the training period: the new different arm is blocked by a baffle plate, and the mice are put from the starting arm and move freely in the starting arm and other arms for 10 min. After the training is ended, the mice are put back into a raising cage. After 20 h, the second stage of the experiment, namely the detection period is started. During the detection period: the baffle plate of the new different arm is removed, and the mice are placed from the starting arm and freely explore and move in the three arms for 5 min. The motion distance of each mouse in each arm within 5 min is recorded by video recording, and the motion distance of the new arm is used as an assessment index.

Rotarod test: The motion coordination ability and the fatigue degree of the mice are assessed. Each mouse is placed on a rotarod fatigue instrument with a rotating speed of 30 rpm/min, and subjected to fatigue rotarod training once a day for 3 consecutive days. A formal rotarod test is conducted. The mice are placed on an electric rotarod fatigue instrument with a rotating speed increasing from 1 rpm/min to 30 rpm/min. The time from the start of the experiment to the drop of the rotarod is recorded as the rotarod residence time, and the length of the residence time is used as an index to assess the states of the mice.

### 3.1.2 Experimental Results:

1) Acid Tolerance of Bifidobacterium animalis subsp. lactis GOLDGUT-BB21:

The data results in Table 1 show that the Bifidobacterium animalis subsp. lactis GOLDGUT-BB21 has good acid tolerance, and the survival rate can reach more than 99% when incubation in an acidic solution at pH of 3.0-4.0 for 3 h.

TABLE 1
Acid tolerance of Bifidobacterium animalis
subsp. lactis GOLDGUT-BB21

| | Survival | Survival | Survival | Average survival |
|---|---|---|---|---|
| pH | rate 1 (%) | rate 2 (%) | rate 3 (%) | rate (%) |
| 3 | 99.58 | 99.76 | 99.62 | 99.65 ± 0.08 |
| 3.5 | 99.18 | 99.26 | 99.89 | 99.44 ± 0.32 |
| 4 | 99.67 | 99.82 | 99.91 | 99.8 ± 0.10 |

2) Alkali Tolerance of Bifidobacterium animalis subsp. lactis GOLDGUT-BB21

The data results in Table 2 show that the Bifidobacterium animalis subsp. lactis GOLDGUT-BB21 has good alkali tolerance, and the survival rate can reach more than 99% when incubation in an alkaline solution at pH of 9.0-10.0 for 4 h.

TABLE 2
Alkali tolerance of Bifidobacterium animalis
subsp. lactis GOLDGUT-BB21

| | Survival | Survival | Survival | Average survival |
|---|---|---|---|---|
| pH | rate 1 (%) | rate 2 (%) | rate 3 (%) | rate (%) |
| 9 | 99.83 | 99.55 | 99.62 | 99.67 ± 0.12 |
| 10 | 99.32 | 99.30 | 99.05 | 99.22 ± 0.12 |

3) Metabolic Fingerprint of Bifidobacterium animalis subsp. lactis GOLDGUT-BB21

The data results in Table 3 show that Bifidobacterium animalis subsp. lactis GOLDGUT-BB21 has a good utilization effect on D-fructose, L-trehalose, etc., can effectively use D-gluconic acid, a-ketovaleric acid, etc., and has a good utilization effect on L-methionine and L-valine in amino acid.

TABLE 3
Metabolic fingerprint of Bifidobacterium animalis subsp. lactis GOLDGUT-BB21

| Substrate | Use | Substrate | Use | Substrate | Use | Substrate | Use |
|---|---|---|---|---|---|---|---|
| Water | − | Dulcitol | − | Glycerin | − | α-methyl-D- | ++ |
| | | | | | | galactoside | |
| N-acetyl-D- | − | i-erythritol | − | D,L-α-glycerol | − | β-methyl-D- | ++ |
| galactosamine | | | | phosphate | | galactoside | |
| N-acetyl-D- | − | D-fructose | ++ | m-inositol | − | α-methyl-D- | ++ |
| glucosamine | | | | | | glucoside | |
| N-acetyl-β-D- | − | L-trehalose | ++ | α-D-lactose | ++ | β-methyl-D- | ++ |
| mannosamine | | | | | | glucoside | |
| Ribitol | − | D-galactose | ++ | Lactulose | + | Palatinose | ++ |
| Amygdalin | ++ | D-galacturonic | ++ | Maltose | ++ | D-raffinose | ++ |
| | | acid | | | | | |
| D-arabitol | − | Gentiobiose | ++ | Maltotriose | ++ | L-rhamnose | ++ |
| Arbutin | ++ | D-gluconic acid | + | D-mannitol | − | Salicin | ++ |
| D-cellobiose | ++ | D-glucosamine | + | D-mannose | ++ | D-sorbitol | − |
| | | acid | | | | | |
| α-cyclodextrin | ++ | α-D-glucose | ++ | D-melezitose | + | Stachyose | ++ |
| β-cyclodextrin | − | Glucose-1- | + | D-melibiose | ++ | Sucrose | ++ |
| | | phosphate | | | | | |
| Dextrin | ++ | Glucose-6- | ++ | 3-methyl-D-glucose | ++ | D-trehalose | − |
| | | phosphate | | | | | |
| Turanose | ++ | Methyl lactic | − | L-alaninamide | − | L-methionine | + |
| | | acid | | | | | |
| Acetic acid | − | D-malic acid | − | L-alanine | − | L-phenylalanine | − |
| Formic acid | − | L-malic acid | − | L-alanyl-L-glutamate | − | L-serine | − |
| Fumaric acid | − | Propionic acid | + | L-propylamino- | − | L-threonine | − |
| | | | | histidine | | | |
| Glyoxylic acid | ++ | Pyruvic acid | ++ | L-alanyl-L-threonine | − | L-valine | + |
| α-hydroxybutyric | − | Methyl pyruvate | ++ | L-asparaginic acid | − | L-valine + | − |
| acid | | | | | | L-aspartate | |
| β-hydroxybutyric | − | D-gluconic acid | − | L-glutamic acid | − | 2′-deoxyadenosine | − |
| acid | | | | | | | |
| Itaconic acid | − | Succinamic acid | − | L-glutamate | − | Inosine | ++ |
| α-ketobutyric acid | + | α-succinic acid | − | Glycine-L-aspartate | − | Thymidine | − |
| α-ketovaleric acid | ++ | Dimethyl | − | Glycyl-L-glutamine | − | Uridine | ++ |
| | | succinate | | | | | |
| D,L-lactic acid | − | m-tartaric acid | − | Glycyl-L-methionine | − | Thymidine | − |
| | | | | | | 5′-monophosphate | |
| L-lactic acid | − | L-urocanic acid | − | Glycyl proline | − | Uridine | − |
| | | | | | | 5′-monophosphate | |

Note:
When Y > 0.1, it indicates that the strain can effectively use this substance, labeled as ++; when 0.1 > Y > 0.05, it indicates that the strain can make good use of this substance, labeled as +; and when Y < 0.05, it indicates that the strain cannot use this substance, labeled as −.

4) Antibacterial Ability of Bifidobacterium animalis subsp. lactis GOLDGUT-BB21:

The data results in Table 4 show that the Bifidobacterium animalis subsp. lactis GOLDGUT-BB21 can effectively inhibit common opportunistic pathogens.

TABLE 4
Antibacterial ability of Bifidobacterium animalis
subsp. lactis GOLDGUT-BB21 (cm)

| Pseudomonas | Staphylococcus | Escherichia |
|---|---|---|
| aeruginosa | aureus | coli |
| 1.2 | 1.1 | 1.1 |

5) Metabolite Detection of Bifidobacterium animalis subsp. lactis GOLDGUT-BB21:

The data results in Table 5 show that the Bifidobacterium animalis subsp. lactis GOLDGUT-BB21 has the ability of high yield of acetic acid and lactic acid.

TABLE 5
Acid production characteristic of Bifidobacterium animalis
subsp. lactis GOLDGUT-BB21 (mg/L)

| Acetic acid | Lactic acid | Succinic acid | Malic acid |
|---|---|---|---|
| 318.80 | 64.33 | 2.62 | 3.21 |

6) Antibiotic Resistance of Bifidobacterium animalis subsp. lactis GOLDGUT-BB21:

The data results in Table 6 show that the Bifidobacterium animalis subsp. lactis GOLDGUT-BB21 is sensitive to many antibiotics, including penicillin, ceftriaxone, etc.

TABLE 6
Detection results of antibiotic sensitivity

| Antibiotic | Re- | Antibiotic | Re- | Antibiotic | Re- |
|---|---|---|---|---|---|
| type | sult | type | sult | type | sult |
| Penicillin | S | Ceftriaxone | S | Midecamycin | S |
| Oxacillin | S | Cefoperazone | S | Norfloxacin | S |
| Ampicillin | S | Amikacin | R | Ofloxacin | S |
| Carbenicillin | S | Gentamicin | R | Ciprofloxacin | S |
| Piperacillin | S | Kanamycin | R | Vancomycin | S |
| Cephalexin | S | Neomycin | R | Polymyxin | R |
| Cefazolin | S | Tetracycline | S | Compound | S |
| | | | | sulfamethoxazole | |
| Cefradine | S | Doxycycline | S | Furazolidone | S |
| Cefuroxime | S | Minocycline | S | Chloramphenicol | S |
| Ceftazidime | S | Erythromycin | S | Clindamycin | S |

Note: R represents resistance, and S represents sensitivity.

7) Improvement of Slow Weight Gain in Mice with CFS by Bifidobacterium animalis subsp. lactis GOLDGUT-BB21:

The results in Table 7 show that the Bifidobacterium animalis subsp. lactis GOLDGUT-BB21 can significantly increase the weight gain of the mice, and the weight gain of the mice in the model group is significantly inhibited.

TABLE 7
Weight of mice in each group (g)

| Mouse No. | No. 1 | No. 2 | No. 3 | No. 4 | No. 5 | Average value | P value | |
|---|---|---|---|---|---|---|---|---|
| Control group | 20.98 | 20.56 | 19.21 | 19.88 | 19.33 | 19.99 ± 0.69 | <0.0001 | |
| Model group | 16.68 | 16.71 | 16.6 | 16 | 16.51 | 16.5 ± 0.26 | | 0.0002 |
| GOLDGUT-BB21 group | 19.04 | 18.45 | 18.2 | 18.26 | 18.17 | 18.42 ± 0.32 | | |

8) Improvement of Memory and Cognitive Functions in Mice with CFS by Bifidobacterium animalis subsp. lactis GOLDGUT-BB21:

The data results in Table 8 and Table 9 show that the mice in the Bifidobacterium animalis subsp. lactis GOLDGUT-BB21 group can significantly improve the spontaneous alternation rate in the Y-maze experiment and significantly increase the motion distance of the new arm in the new arm recognition experiment, which indicates that the Bifidobacterium animalis subsp. lactis GOLDGUT-BB21 can improve the spatial memory impairment of the mice with CFS.

TABLE 8
Spontaneous alternation rate of model mice

| Mouse No. | No. 1 | No. 2 | No. 3 | No. 4 | No. 5 | Average value | P value | |
|---|---|---|---|---|---|---|---|---|
| Control group | 62.16 | 51.85 | 56.52 | 55.17 | 60 | 57.14 ± 3.62 | <0.0001 | |
| Model group | 37.14 | 37.5 | 27.27 | 33.33 | 38.5 | 34.75 ± 4.13 | | 0.0001 |
| GOLDGUT-BB21 group | 58.62 | 59.09 | 65.63 | 45.45 | 55.56 | 56.87 ± 6.58 | | |

TABLE 9
Motion distance of model mice in new arm (cm)

| Mouse No. | No. 1 | No. 2 | No. 3 | No. 4 | No. 5 | Average value | P value | |
|---|---|---|---|---|---|---|---|---|
| Control group | 548.87 | 663.26 | 554.47 | 778.28 | 558.69 | 620.71 ± 89.48 | 0.0002 | |
| Model group | 437.48 | 260.08 | 284.2 | 286.31 | 310.1 | 315.63 ± 62.95 | | 0.0002 |
| GOLDGUT-BB21 group | 630.15 | 566.58 | 588.66 | 741 | 564.41 | 618.16 ± 65.81 | | |

9) Improvement of Anxiety State of Model Mice by Bifidobacterium animalis subsp. lactis GOLDGUT-BB21:

The results in Table 10 and Table 11 show that the Bifidobacterium animalis subsp. lactis GOLDGUT-BB21 can significantly increase the motion distance of the model mice in the open arm and the number of entry in the open arm, and alleviate the anxiety state of the mice with IBS.

TABLE 10
Time to enter open arm for mice in each group after intervention (s)

| Mouse No. | No. 1 | No. 2 | No. 3 | No. 4 | No. 5 | Average value | P value | |
|---|---|---|---|---|---|---|---|---|
| Control group | 22.56 | 34.44 | 30.16 | 15.24 | 18.81 | 24.24 ± 7.10 | 0.0006 | |
| Model group | 4.84 | 7.08 | 6.44 | 7.31 | 7.22 | 6.58 ± 0.92 | | 0.002 |
| GOLDGUT-BB21 group | 17.32 | 21.09 | 19.81 | 21.44 | 28.52 | 21.64 ± 3.73 | | |

TABLE 11
Motion distance of mice of each group in open arm

| Mouse No. | No. 1 | No. 2 | No. 3 | No. 4 | No. 5 | Average value | P value | |
|---|---|---|---|---|---|---|---|---|
| Control group | 106.84 | 90.84 | 44.82 | 60.23 | 86.01 | 77.75 ± 22.26 | 0.0003 | |
| Model group | 25.41 | 21.34 | 10.06 | 29.36 | 14.22 | 20.08 ± 7.08 | | 0.001 |
| GOLDGUT-BB21 group | 84.01 | 81.2 | 60.34 | 59.59 | 70.46 | 71.12 ± 10.17 | | |

10) Improvement of Motion Ability in Mice with CFS by Bifidobacterium animalis subsp. lactis GOLDGUT-BB21:

The results in Table 12 show that the Bifidobacterium animalis subsp. lactis GOLDGUT-BB21 can significantly prolong the rotarod residence time of the mice in the rotarod experiment, which indicates that the Bifidobacterium animalis subsp. lactis GOLDGUT-BB21 has an alleviating effect on the fatigue symptoms of the mice with CFS.

TABLE 12
On-rod time of model mice

| Mouse No. | No. 1 | No. 2 | No. 3 | No. 4 | No. 5 | Average value | P value | |
|---|---|---|---|---|---|---|---|---|
| Control group | 420 | 420 | 402.19 | 420 | 420 | 416.44 ± 7.12 | <0.0001 | |
| Model group | 81.04 | 118.06 | 136 | 126.84 | 49.48 | 102.28 ± 32.34 | | <0.0001 |
| GOLDGUT-BB21 group | 325.21 | 420 | 325.78 | 420 | 420 | 382.20 ± 46.30 | | |

3.2 Animal Experiment on Alleviating IBS by Bifidobacterium animalis subsp. lactis GOLDGUT-BB21

### 3.2.1 Materials and Methods:

1) Improved MRS Liquid Medium (g/L):

The modified MRS liquid medium comprises 52.4 g of MRS broth, 0.01 g of hemin, 0.001 g of resazurin and 10% (v/v) of clarified rumen fluid; distilled water is added to 1 L; pH is 7.2±0.1; and the medium is sterilized at 115° C. for 25 min, and then used after high pressure sterilization. 15 g of additional agar is added to a solid medium.

### 2) Preparation Method of GOLDGUT-BB21 Powder Feed

The Bifidobacterium animalis subsp. lactis is activated twice in the modified MRS broth medium, then inoculated into a modified MRS liquid medium with the same composition at an inoculation amount of 0.1%-10%, and cultured anaerobically at 37° C. for 2 days. The bacterial solution is centrifuged at 4° C. and 10000 rpm for 2 minutes to collect the precipitated cells; the cells are washed with PBS, then suspended by 5:1 in a solution containing 15%-25% of skimmed milk powder, freeze-dried, and tested for viable count.

### 3) Design of Animal Experiment

18 SPF wild female C57BL/6J mice are adopted in the experiment, randomly divided into 3 groups with 6 mice in each group, and adaptively fed for one week.

On days 1-5 of the experiment, each mouse in GOLDGUT-BB21 group is given mixed quantitative bacterial meal feed (viable count 2×10⁹ CFU/day/mouse); and the mice in the control group and the model group are given aseptic meal feed every day.

On days 6-15 after the start of the experiment, all the mice except those of the control group undergo water avoidance stress (WAS) stimulation, and the mice in the control group undergo false water avoidance stress stimulation.

Endpoint sampling: a. colon

### 4) Index Detection

a) Behavioral detection: elevated plus maze test;
b) Colorectal distension experiment

On day 20 of the experiment, after fasting for 12 h without water deprivation, the visceral sensitivity of the mice is measured by abdominal withdrawal reflex (AWR) caused by colorectal distension (CRD).

After the mice are anesthetized by intraperitoneal injection of urethane, a catheter coated with paraffin oil is inserted into the colorectum of the mice. The tail of the balloon is 1 cm away from the anus. The catheter is fixed to the tail of the mice with an adhesive tape. The mice adapt for 20 min after waking up.

The tail of the catheter is connected with a syringe through a tee tube. The colorectal distension is performed with gas pressure values of 0.05, 0.1, 0.15 and 0.2 ml respectively. The target pressure is reached within 3 s, and maintained for 20 s. The stimulation interval is 4 min. Under unknown pressure, the AWR of the mice is observed and scored. Three repetitions are made for each pressure. AWR scores are used to evaluate the visceral sensitivity of the mice.

Scoring criteria: score 0: no obvious response to distension; score 1: except for shaking of the mouse head at the beginning of the stimulation, there is no obvious contraction of the abdominal wall of the mice during the rectal stimulation; score 2: slight abdominal muscle contraction occurs in the mice during stimulation, but the abdomen does not lift; score 3: during stimulation, the abdominal muscles of the mice are contracted strongly, and the abdomen can leave the platform and lift up; score 4: during stimulation, the abdominal muscles of the mice show the strongest contraction, the body is arched and the pelvis leaves the platform and lifts up.

### 3.2.2 Experimental Results:

1) Improvement of Weight Growth Rate of Mice with IBS by Bifidobacterium animalis subsp. lactis GOLDGUT-BB21:

The results in Table 13 show that the Bifidobacterium animalis subsp. lactis GOLDGUT-BB21 can significantly increase the weight growth rate of the mice, and the weight growth of the mice in the model group is significantly inhibited.

TABLE 13
Net weight growth rate of mice in each group (%)

| Mouse No. | No. 1 | No. 2 | No. 3 | No. 4 | No. 5 | Average value | P value | |
|---|---|---|---|---|---|---|---|---|
| Control group | 8.61 | 6.31 | 5.18 | 6.7 | 10.01 | 7.36 ± 1.57 | <0.0001 | |
| Model group | −1.8 | 0.39 | 0.6 | 0.26 | −0.9 | 0.29 ± 0.84 | | <0.0001 |
| GOLDGUT-BB21 group | 8.88 | 7.11 | 8.02 | 8.74 | 6.98 | 7.95 ± 0.72 | | |

2) Improvement of Visceral Hypersensitivity in Mice with IBS by Bifidobacterium animalis subsp. lactis GOLDGUT-BB21:

The data results in Table 14 show that the Bifidobacterium animalis subsp. lactis GOLDGUT-BB21 can significantly reduce the pain response and significantly improve the symptoms of visceral hypersensitivity for the mice in the model group.

TABLE 14
Pain evaluation scores of model mice

| Groups | No. | 0.05 ml | 0.1 ml | 0.15 ml | 0.2 ml |
|---|---|---|---|---|---|
| Control group | No. 1 | 0 | 1.5 | 2.5 | 4 |
| | No. 2 | 0 | 0 | 1.5 | 3 |
| | No. 3 | 1 | 1 | 2 | 4 |
| | No. 4 | 0.5 | 1 | 2.5 | 4 |
| | No. 5 | 0 | 0 | 2 | 3.5 |
| Model group | No. 1 | 0 | 2 | 3 | 4 |
| | No. 2 | 1 | 2.5 | 3.5 | 4 |
| | No. 3 | 1 | 2.5 | 3 | 4 |
| | No. 4 | 1 | 2.5 | 3.5 | 4 |
| | No. 5 | 0 | 2 | 3 | 4 |
| GOLDGUT-BB21 | No. 1 | 0 | 0.5 | 1.5 | 4 |
| group | No. 2 | 0.5 | 1.5 | 2.5 | 4 |
| | No. 3 | 0.5 | 1.5 | 2.5 | 4 |
| | No. 4 | 0.5 | 1 | 2 | 4 |
| | No. 5 | 0 | 1 | 2 | 3 |

3) Improvement of Anxiety State of Model Mice by Bifidobacterium animalis subsp. lactis GOLDGUT-BB21:

The results in Table 15 and Table 16 show that the Bifidobacterium animalis subsp. lactis GOLDGUT-BB21 can significantly increase the motion distance of the model mice in the open arm and the number of entry in the open arm in the elevated plus maze test, and alleviate the anxiety state of the mice with IBS.

TABLE 15
Motion distance of mice of each group in open arm after intervention (cm)

| Mouse No. | No. 1 | No. 2 | No. 3 | No. 4 | No. 5 | Average value | P value | |
|---|---|---|---|---|---|---|---|---|
| Control group | 37.93 | 37.5 | 43.75 | 31.58 | 39.39 | 38.03 ± 3.91 | <0.0001 | |
| Model group | 17.24 | 21 | 11.11 | 23.08 | 13.16 | 17.12 ± 4.53 | | <0.0001 |
| GOLDGUT-BB21 group | 29.09 | 34.48 | 35.71 | 36 | 35 | 34.06 ± 2.54 | | |

TABLE 16
Number of entry in open arm for mice of each group after intervention (%)

| Mouse No. | No. 1 | No. 2 | No. 3 | No. 4 | No. 5 | Average value | P value | |
|---|---|---|---|---|---|---|---|---|
| Control group | 6.83 | 7.07 | 7.91 | 7.15 | 11.14 | 8.02 ± 1.60 | 0.004 | |
| Model group | 0.57 | 2.85 | 0.84 | 1.48 | 5.34 | 2.22 ± 1.75 | | 0.006 |
| GOLDGUT-BB21 group | 5.39 | 6.35 | 7.41 | 7.93 | 4.9 | 6.40 ± 1.15 | | |

4) Significant Increase in Expression of Claudin-Related Gene in Colon by Bifidobacterium animalis subsp. lactis GOLDGUT-BB21:

The results in Table 17 show that the intervention of the Bifidobacterium animalis subsp. lactis GOLDGUT-BB21 can significantly increase the expression level of the claudin-related gene ZO-1 in the colon of the mice with IBS, significantly enhance the intestinal wall integrity of the mice with IBS, and significantly improve the barrier function of the intestinal wall.

TABLE 17
Expression level of gene ZO-1 in colon of mice after intervention of strain

| Mouse No. | No. 1 | No. 2 | No. 3 | No. 4 | No. 5 | Average value | P value | |
|---|---|---|---|---|---|---|---|---|
| Control group | 1.63 | 2.26 | 1.53 | 1.54 | 1.61 | 1.71 ± 0.28 | 0.03 | |
| Model group | 1.09 | 1.24 | 0.92 | 1.23 | 0.91 | 1.08 ± 0.14 | | 0.02 |
| GOLDGUT-BB21 group | 1.72 | 2.44 | 2.04 | 1.36 | 1.33 | 1.78 ± 0.42 | | |

### REFERENCES

1. Chu L.Beyond Myalgic Encephalomyelitis/Chronic Fatigue Syndrome: Redefining an Illness. 2015.
2. Carruthers B M, van de Sande M I, De Meirleir K L, et al. Myalgic encephalomyelitis: international consensus criteria. J Intern Med. 2011;270:327-338.
3. Chey W D, Kurlander J, Eswaran S. Irritable bowel syndrome: a clinical review. JAMA. 2015;313:949-958.
4. Canavan C, West J, Card T. Review article: the economic impact of the irritable bowel syndrome. Aliment Pharmacol Ther. 2014;40:1023-1034.
5. G. Burns, G. Carroll, A. Mathe, et al., Evidence for local and systemic immune activation in functional dyspepsia and the irritable bowel syndrome: a systematic review, Am. J. Gastroenterol. 2019; 114:429-436.

What is claimed is:

1. A Bifidobacterium animalis subsp. Lactis GOLDGUT-BB21, with a preservation number of CGMCC No.29347

2. A microbial agent, comprising an active ingredient of the Bifidobacterium animalis subsp. Lactis GOLDGUT-BB21 according to claim 1.

3. A composition, comprising the Bifidobacterium animalis subsp. Lactis GOLDGUT-BB21 according to claim 1.

4. An application of the Bifidobacterium animalis subsp. lactis GOLDGUT-BB21 according to claim 1 in preparation of health food or drugs for treating or improving chronic fatigue syndrome.

5. The application according to claim 4, wherein the chronic fatigue syndrome comprises: symptoms of inhibited weight gain, impaired spatial memory, and impaired motion ability.

6. An application of the Bifidobacterium animalis subsp. lactis GOLDGUT-BB21 according to claim 1 in preparation of health food or drugs for treating or improving irritable bowel syndrome.

7. The application according to claim 6, wherein the irritable bowel syndrome comprises symptoms of inhibited weight gain, visceral hypersensitivity, anxiety, and intestinal wall barrier dysfunction.

8. An application of the Bifidobacterium animalis subsp. lactis GOLDGUT-BB21 according to claim 1 in preparation of bacterial agents for inhibiting Escherichia coli, Staphylococcus aureus or Pseudomonas aeruginosa.
